(19) United States
(12) Patent Application Publication
(10) Pub. No.: US 2004/0170594 A1
(43) Pub. Date: Sep. 2, 2004

(54) Hair lotion stimulating hair growth

(76) Inventor: Ahmed Kadri, Marseille (FR)

Correspondence Address: GREENBLUM & BERNSTEIN, P.L.C., 1950 ROLAND CLARKE PLACE, RESTON, VA, 20191, US

(21) Appl. No.: 10/481,885

(22) PCT Filed: Jul. 3, 2002
(86) PCT No.: PCT/FR02/02314

(30) Foreign Application Priority Data
Jul. 3, 2001 (FR) 01/08795

(51) Int. Cl. A61K 35/78; A61K 7/06

(52) U.S. Cl. 424/74; 424/736; 424/754

(57) ABSTRACT

The invention concerns a hair lotion stimulating hair growth. It consists of a mixture comprising at least the following constituents: iodised salt, lemon juice, fresh onion, citric acid and alcohol, a fragrance designed to neutralise the onion smell being preferably added to said mixture. Said lotion concerns the industrial and commercial field of production and commercial distribution of hair care products, and is suitable for both women and men, independently of their age.

The present invention relates to a hair lotion stimulating hair growth.

It relates to the industrial and commercial field of production and distribution of hair care products, and is suitable for both women and men, independently of their age.

Hair has always been very important for the appearance of women and men.

Thus, a shaven head is generally considered as a sign of grief and sorrow, as evoked in numerous ancient texts showing that women shaved their head when tragedy occurred in their family or city.

Conversely, having long hair is a sign of power as shown in the Bible through the story of Samson and Delilah. For the Franks, it was also the sign of royal dignity.

Hair loss, due mainly to aging, but which can also be caused by other factors such as certain diseases, is often felt almost as an infirmity, and many people incur substantial expenses in an attempt to remedy the situation, trying, unsuccessfully most of the time, to suppress or at least to hide their baldness.

There are numerous commercially available products advertised as capable of causing hair growth. To our knowledge, while some of them can indeed slow hair loss momentarily, none of them can cause hair to grow back. Moreover, most of these products contain a large amount of fatty bodies, which makes their use unpleasant and hairstyling unaesthetic as hair becomes sticky.

The object of the invention is to overcome these drawbacks by proposing a product that is completely free of fatty bodies, which not only stops hair loss but also ensures an effective and visible hair growth, as shown by a series of tests performed on more than one hundred people.

The product is constituted of a mixture of the following constituents: iodized salt, lemon juice, fresh onion, citric acid and alcohol, a fragrance capable, of neutralizing the onion smell being preferably added to this mixture.

The following detailed description relates to non-limiting examples of embodiment of the object of the present patent application.

The hair lotion according to the invention is a 100% natural product with no fatty matters.

It is constituted of a mixture containing at least the following substances:

Iodized salt

Lemon juice

Fresh onion

Citric acid

Alcohol.

Because of the unpleasant onion smell, one can advantageously add a predetermined fragrance to neutralize this smell, or replace the alcohol with a scented alcoholate.

The constituents of the mixture are apportioned in quantities approximate to the following values.

| | |
|---|---|
| Iodized salt | 10% |
| Lemon juice | 15% |
| Fresh onion | 50% |
| Citric acid | 5% |
| Alcohol or | 20% |
| Scented alcoholate | |

Due to a very low cost given the inexpensive ingredients used, the product that has just been described lends itself particularly well to the manufacture of hair lotions adapted for hair growth, and for which a very large scale distribution can be envisioned.

The positioning of the various constituent elements give the object of the invention a maximum of useful effects which, until now, had not been achieved by similar products.

1. Hair lotion stimulating hair growth, adapted to hair care and suitable for both women and men, independently of their age,
characterized in that it is composed of a mixture containing iodized salt, lemon juice, fresh onion, citric acid, and alcohol.

2. Hair-lotion according to claim 1, characterized in that it further contains a fragrance capable of neutralizing the onion smell.

3. Hair lotion according to claim 1, characterized in that alcohol is replaced with a scented alcoholate capable of neutralizing the onion smell.

4. Hair lotion according to any of the preceding claims, characterized in that the constituents of the mixture are apportioned in quantities close to: 10% of iodized salt, 15% of lemon juice, 50% of fresh onion, 5% of citric acid, and 20% of alcohol or scented alcoholate.
